(19) United States
(12) Patent Application Publication
(10) Pub. No.: US 2020/0087008 A1
(43) Pub. Date: Mar. 19, 2020

(54) Method to Distribute a Single Use Podiatry Bur

(71) Applicant: Bharat Bihani, Jacksonville, FL (US)

(72) Inventor: Bharat Bihani, Jacksonville, FL (US)

(21) Appl. No.: 16/134,349

(22) Filed: Sep. 18, 2018

(51) Int. Cl. B65B 5/04; A61B 17/54; A61B 50/30

(52) U.S. Cl. CPC B65B 5/04; A61B 2017/0023; A61B 50/30; A61B 17/54

(57) ABSTRACT

This podiatry bur will be delivered in a single use package and will be delivered to the provider and then used and then discarded. Typically, the bur that is used today is oftentimes cleaned between uses. Unfortunately, the cleaning process leaves particles of dead skin and other debris (residual protein) in the bur cavities, which are impossible to clean making their continued use less than ideal.

### FIELD OF THE INVENTION

This is a method to prepackage a single use podiatry bur; the word “bur” may also be spelled as “burr” and this application is intended to cover all types of podiatry burs. Podiatry burs are used to treat aliments of the feet, which can be cover a variety of issues. There are many people who suffer from conditions such as diabetes that warrant foot care a primary importance. Diabetes is just one of the many conditions that must be addressed when discussing foot care for those individuals who are “at risk”. In the prior art the podiatry bur is shipped in a lot with no specific means to sterilize the bur. This technique increases the risk of injury and infection to those persons who are already at risk. This application seeks to eliminate the risk of injury/infection with the use of a single package, pre-sterilized bur that is disposable.

### PRIOR ART

Burs are used are frequently found in the field of dentistry to treat problems with the mouth. This type of bur is placed in a person's mouth and the dentist may clean the bur or discard the bur. If the bur is cleaned certain particles (residual protein) may remain on the bur and may be introduced into the mouth of the next patient in the case of a dental bur. With this application the type of bur that is used on a person's foot is pre-sterilized and is intended to be discarded. In the prior art certain attempts may be made to clean the bur with the same result: residual protein. This, of course, is not ideal as the next patient may be subjected to another person's skin. In this application the bur is pre-sterilized and after it is used is discarded to prevent the problem of contamination for the next patient.

### BRIEF SUMMARY OF THE INVENTION

Currently podiatry burs are used to remove excess skin and debris from the surfaces of feet. There are certain individuals who are “at risk” for issues related to the feet. While diabetic patients are not the only group of people who are “at risk” this application will discuss this patient to demonstrate the unique value of this concept.

The health care professional will select a particular podiatry bur for a medical problem for that individual who is at risk. In the case of a diabetic patient diabetes can affect a person's feet. Another unique challenge for the diabetic patient is the longer recovery period because of the diabetes. It is also important for the diabetic patient to minimize the chance of injury to the person's feet to prevent any delay in recovery.

There are a multitude of shapes of podiatry burs that are used to treat problems related to the person's feet. There are also a multitude of types of burs that can be used for a particular problem. This application covers all shapes and types of podiatry burs.

As stated before, in the prior art, the bur, which has a variety of irregular shapes, is used and then cleaned by the medical provider. This is an imperfect process as debris (residual protein) may be left in one or more of the cavities of the burs. While some of the residual protein may be visible to the naked eye, other type of residual protein may not be visible to the naked eye. In the case of residual protein that is visible to the naked eye, a human must closely examine the surfaces of the bur to determine whether or not any residual protein is visible; because of the human factor, errors in the detection process will occur and some residual protein will be left on the bur. In the case of microscopic matter, there is no practical way to make the detection complete. This method of cleaning is obviously imperfect and may then lead to increased infection rates and sub-standard cleaning for subsequent uses.

While no one wants to deliberately injure a person, the risk of injury is increased with these types of sterilization techniques. If the person is injured with the introduction of residual protein, the risk of further complications may be increased. In the example of the diabetic patient, any delay in the recovery process increases the cost of caring for that patient. The cost of caring for a patient dwarfs the cost of the appropriate bur that is ten discarded.

Sterilization techniques are many and no specific sterilization technique is being claimed with this application. However, the bur when shipped in this application is pre-sterilized in a single package and after it is used, is disposed.

### BRIEF DESCRIPTION OF THE DRAWINGS

FIGURES are not needed with this application.

### DETAILED DESCRIPTION OF THE EMBODIMENTS

The use of podiatry burs are very common in the prior art. The podiatry bur is used for the cleaning of feet and the surfaces of the foot for those patients who are “at risk”. Podiatry burs come in various shapes and sizes depending on the specific use that is anticipated. For instance a representative podiatry bur can be shaped like a diamond olive shape, diamond large barrel shape, a diamond bud shape, a safe toe round edge grinder as well as a diamond small taper; this is not an exhaustive list but merely representative of the shapes of podiatry burs. Additionally, different materials may also be used in the podiatry burs. For instance, the use of stainless steel which is diamond coated as well as different types of carbide or ceramic burs may be used. Additionally, the family of carbide burs may be further broken down into carbide fine, carbide coarse, or carbide medium burs. This list of the types of materials is not an exhaustive list but merely representative. It is also anticipated that different materials may be developed and the different types of materials will be covered by this application. It is not the shape of the bur or the type of material that is important but rather it is the concept of a single use podiatry bur that is used and then discarded that is central to this application.

The podiatry bur is used to treat infections or medical issues that are related to the feet such as diabetic effected feet, ingrown toenails or a myriad of other medical conditions of those persons who are “at risk”.

The specific podiatry bur that is used is a decision that is left to the medical professional and is selected based on the condition that needs to be addressed as well as the particular material of the bur itself. The selection of a bur must be made based on the physical condition of the patient and his or her medical condition but this decision is left to the health care professional.

In this application the bur is sterilized at a remote location and then shipped in a single package to the provider in its own sterile packaging. The bur will remain in its sterile environment until the packaging is opened prior to the bur's use. Once the bur packaging has been opened, the bur must be used on the patient.

The bur is used on the individual patient and is then discarded. The use of a single disposal bur eliminates any chance that the person can be infected with a bur that has been cleaned imperfectly. If the patient does require additional care because of an imperfect or poorly cleaned bur the cost of caring for such an “at risk” patient is much greater than the cost of the disposable bur, which is contemplated in this application.

The bur is meant to be discarded and is not intended to be reused. The exact disposal means for the bur may vary but all disposal should be accomplished according to industry standards to protect the patient as well as the health care professional.

The inventor claims:

1. A method to distribute a podiatry bur which is comprised of the following steps:
a. selection of the podiatry bur;
b. sterilizing the podiatry bur;
c. individually wrapping the podiatry bur at a remote location;
d. shipping the individually wrapped podiatry bur;
e. unwrapping the podiatry bur;
f. using the podiatry bur;
g. discarding the podiatry bur,

2. The method to distribute a podiatry bur as described in claim 1 wherein the bur can treat diabetes effected feet.

3. The method to distribute a podiatry bur as described in claim 1 wherein the bur can treat toenails.

4-8. (canceled)
